(19) United States
(12) Patent Application Publication
(10) Pub. No.: US 2008/0021501 A1
(43) Pub. Date: Jan. 24, 2008

(54) High strength suture with UHMWPE and polybutester

(75) Inventor: John W. Schmieding, Naples, FL (US)

Correspondence Address: DICKSTEIN SHAPIRO LLP, 1825 EYE STREET NW, Washington, DC, 20006-5403, US

(73) Assignee: Arthrex, Inc.

(21) Appl. No.: 11/822,566

(22) Filed: Jul. 6, 2007

Related U.S. Application Data
(60) Provisional application No. 60/819,002, filed on Jul. 7, 2006.

(51) Int. Cl. A61B 17/04; A61L 17/10

(52) U.S. Cl. 606/228; 428/364; 428/371; 428/394

(57) ABSTRACT

A high strength abrasion resistant surgical suture material with improved tie down characteristics. The suture features a multifilament cover formed of strands of ultra high molecular weight long chain polyethylene braided with polybutester. Selected nylon fibers in the cover may be provided in a color contrasting with the other cover fibers to provide an identifiable trace. The cover surrounds a core formed of twisted strands of ultrahigh molecular weight polyethylene. The suture, provided in a #2 size, has the strength of #5 Ethibond, is ideally suited for most orthopedic procedures, and can be attached to a suture anchor or a curved needle.

### CROSS-REFERENCE TO RELATED APPLICATIONS

This application claims priority to U.S. Provisional Application No. 60/819,002, filed on Jul. 7, 2006, the entire contents of which are hereby incorporated by reference.

### BACKGROUND OF THE INVENTION

1. Field of the Invention

The present invention relates to a high strength surgical suture, and more particularly to braided suture blends of ultrahigh molecular weight polyethylene (UHMWPE) having high strength and excellent tie down characteristics.

2. Description of the Related Art

Suture strength is an important consideration in any surgical suture material. One of the strongest materials currently formed into elongated strands is an ultrahigh molecular long chain weight polyethylene, typically used for fishing line and the like, which is sold under the trade names Dyneema or Spectra. This material is much stronger than ordinary surgical suture; however, it has poor knot tie down characteristics for use in surgical applications.

### SUMMARY OF THE INVENTION

The present invention advantageously provides a high strength surgical suture with improved tie down characteristics. The suture features a braided cover made of a blend of ultrahigh molecular weight long chain polyethylene fiber and a fiber of one or more long chain synthetic polymers, preferably polybutester. The ultrahigh molecular weight polyethylene provides strength. The polybutester provides improved tie down properties.

Handling properties of the high strength suture also are enhanced using various materials to coat the suture. In addition, strands of a contrasting color may optionally be added to the braided threads to enhance visibility. The colored strands preferably are dyed filaments of polybutester or nylon.

In a preferred embodiment, the suture includes a multifilament cover formed of ultrahigh molecular weight polyethylene fiber braided with polybutester and nylon fibers. The cover surrounds a fiber core substantially or entirely of ultrahigh molecular weight polyethylene. The core preferably comprises 3 strands of ultrahigh molecular weight polyethylene, twisted at about 3 to 6 twists per inch.

The cover preferably comprises 8 strands of ultrahigh molecular weight polyethylene braided with 6 strands of polybutester and 2 strands of nylon, the nylon strands being provided in black or some other contrasting color as explained in greater detail below.

Ultrahigh molecular weight polyethylene fibers suitable for use in the present invention are marketed under the Dyneema trademark by Toyo Boseki Kabushiki Kaisha, and are produced in the U.S. by Honeywell under the trademark Spectra.

The suture of the present invention advantageously has the strength of Ethibond #5 suture, yet has the diameter, feel and tie ability of #2 suture. As a result, the suture of the present invention is ideal for most orthopedic procedures such as rotator cuff repair, achilles tendon repair, patellar tendon repair, ACL/PCL reconstruction, hip and shoulder reconstruction procedures, and replacement for suture used in or with suture anchors. The suture can be packaged ready for use attached to a suture anchor.

The suture can be uncoated, or coated with wax (beeswax, petroleum wax, polyethylene wax, or others), silicone (Dow Corning silicone fluid 202A or others), silicone rubbers (Nusil Med 2245, Nusil Med 2174 with a bonding catalyst, or others) PTFE (Teflon, Hostaflon, or others), PBA (polybutylate acid), ethyl cellulose (Filodel) or other coatings, to improve lubricity of the braid, knot security, or abrasion resistance, for example. As an added advantage, as mentioned above, all or some of the nylon fibers (or the polybutester fibers) in the cover are provided in a contrasting color for visibility and identification purposes. A few trace threads having a contrasting color, preferably of a readily dyed fiber such as polybutester or nylon, in the cover aid surgeons in identifying the travel direction of the suture during surgery, particularly during arthroscopic operations. Providing the trace threads in a regularly repeating pattern is particularly useful, allowing the surgeon to distinguish different ends of lengths of suture, and determine the direction of travel of a moving length of suture. Of the more easily dyed fibers, nylon is preferred in that it accepts dye readily.

Other features and advantages of the present invention will become apparent from the following description of the invention which refers to the accompanying drawings.

### BRIEF DESCRIPTION OF THE DRAWINGS

FIG. 1 is a copy of a scanning electron micrograph of a length of suture according to the present invention.

FIG. 2 is a schematic cross section of a length of suture according to the present invention.

FIG. 3 is an illustration of the suture of the present invention attached to a suture anchor.

FIGS. 4A and 4B show the suture of the present invention attached to a half round, tapered needle.

### DETAILED DESCRIPTION OF THE PREFERRED EMBODIMENT

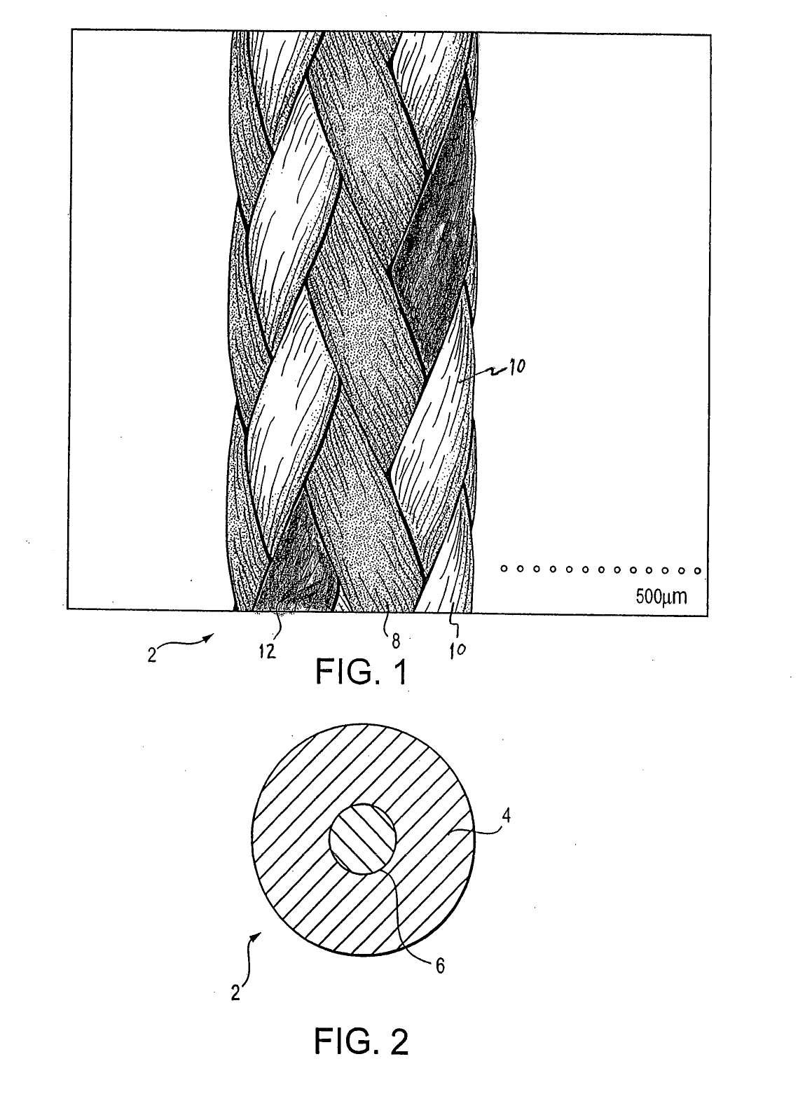

Referring to FIG. 1, a scanning electron micrograph of a length of suture 2 according to the present invention is shown. Suture 2 is made up of a cover 4 and a core 6 surrounded by the cover. See FIG. 2. Strands of ultrahigh molecular weight polyethylene (UHMWPE) 8, sold under the tradename Spectra or Dyneema, and strands of polybutester 10 and 12 are braided together to form the cover 4. Core 6 is formed of twisted strands of UHMWPE.

Polybutester (Novofil) is made of a copolymer of polyglycol terephthalate and polytrimethylene terephthalate. Polybutester is very elastic and has a very low coefficient of friction. These properties are ideal for surface closure, permitting adequate tissue approximation while allowing for tissue edema and detumescence. Polybutester does not lose tensile strength or become absorbed. Polybutester (PBE) suture may be coated with an absorbable polytribolate polymer that is composed of glycolide (9%), epsilon-caprolactone (51%), and poloxamer 188 (40%) to reduce its drag forces. Coated and uncoated polybutester sutures exhibit less stiffness than polypropylene sutures and exhibit limited memory after removal from the suture package. Coating the polybutester suture reduces the drag forces in musculoaponeurotic, colonic, and vascular tissues.

UHMWPE strands 8 are substantially translucent or colorless. The majority of the polybutester strands 10 are white (undyed), with one or more additional polybutester or nylon strands 12 having a contrasting color provide a trace in the suture. Due to the transparent nature of the UHMWPE, the suture takes on the color of strands 10 and 12, and thus appears to be white with a trace in the contrasting color. In accordance with the present invention, trace strands 12 are preferably provided in black. The black trace assists the surgeon in differentiating between suture strands with the trace and suture strands without the trace. The trace also assists the surgeon in identifying whether the suture is moving.

Details of the present invention will be described further below in connection with the following examples:

### EXAMPLE 1

### USP Size 5 (EP Size 7)—Without Nylon Trace

Made on a 16 carrier Hobourns machine, the yarns used in the braided cover are Honeywell Spectra 2000, polyester type 712. The cover is formed using eight carriers (strands) polybutester type 712 and Spectra. The cover is formed using eight carriers (strands) with one end of 190 decitex polybutester per carrier, and eight carriers with one end of 220 decitex Spectra per carrier. The core is formed of Spectra using one end of 440/1/3/ twisted 10 tpi “z” and 7 tpi “s” (core is not steam set). Picks per inch (PPI)=36. In forming the suture, the percent cover is 71.31, while the percent core is 28.69 (by weight). Runnage is 1991 meters per kilo.

Of the overall suture, the polybutester in the cover (8 carriers×190 decitex=1520 decitex) makes up 33% of the suture, and the Spectra in the cover (8 carriers×220 decitex=1760 decitex) makes up 38.76% of the suture. The Spectra core (3 carriers×440 decitex=1320 decitex) is 28.69% of the suture.

### EXAMPLE 2

### USP Size 5 (EP Size 7)—With Nylon Trace

Made on a 16 carrier Hobourns machine, the yarns used in the braided cover are Honeywell Spectra 2000, polybutester type 712, and nylon. The cover is formed using eight strands of 144 decitex Spectra per carrier, braided with six strands of 100 decitex polybutester, and two strands of colored nylon. The core is formed of three carriers of 144 decitex Spectra braided at three to six twists per inch.

### EXAMPLE 3

### USP Size 2

The suture is 38.09% polybutester, 61.91% UHMWPE, or about 40% polybutester and about 60% UHMWPE, by weight.

The examples set forth above are for size 2 and size 5 suture. In the making of various sizes of the inventive suture, different decitex values and different PPI settings can be used to achieve the required size and strength needed. In addition, smaller sizes may require manufacture on 12 carrier machines, for example. The very smallest sizes preferably are made without a core. Overall, the suture may range from 5% to 90% (by weight) ultrahigh molecular weight polymer (preferably at least 40% ultrahigh molecular weight polymer), with the balance formed of polybutester and/or nylon. The core preferably comprises 18% or greater of the total amount (by weight) of filament.

The suture preferably is coated with wax (beeswax, petroleum wax, polyethylene wax, or others), silicone (Dow Corning silicone fluid 202A or others), silicone rubbers (Nusil Med 2245, Nusil Med 2174 with a bonding catalyst, or others) PTFE (Teflon, Hostaflon, or others), PBA (polybutylate acid), ethyl cellulose (Filodel) or other coatings, to improve lubricity of the braid, knot security, or abrasion resistance, for example.

The ultra high molecular weight (UHMW) polymer component of the present invention provides strength, and the polybutester component is provided to improve tie ability and tie down characteristics. However, it has been found that the UHMW polymer provides an unexpected advantage of acting as a cushion for the polybutester fibers, which are relatively hard and tend to damage each other. The UHMW polymer prevents breakage by reducing damage to the polybutester when the suture is subjected to stress.

According to an alternative embodiment of the present invention, a partially bioabsorbable suture is provided by blending a high strength material, such as UHMWPE fibers, with a bioabsorbable material, such as PLLA or one of the other polylactides, for example. Accordingly, a suture made with about 10% Spectra or Dyneema blended with absorbable fibers would provide greater strength than existing bioabsorbable suture with less stretch. Over time, 90% or more of the suture would absorb, leaving only a very small remnant of the knot. The absorbable suture can include coatings and colored traces as noted above for nonabsorbable suture.

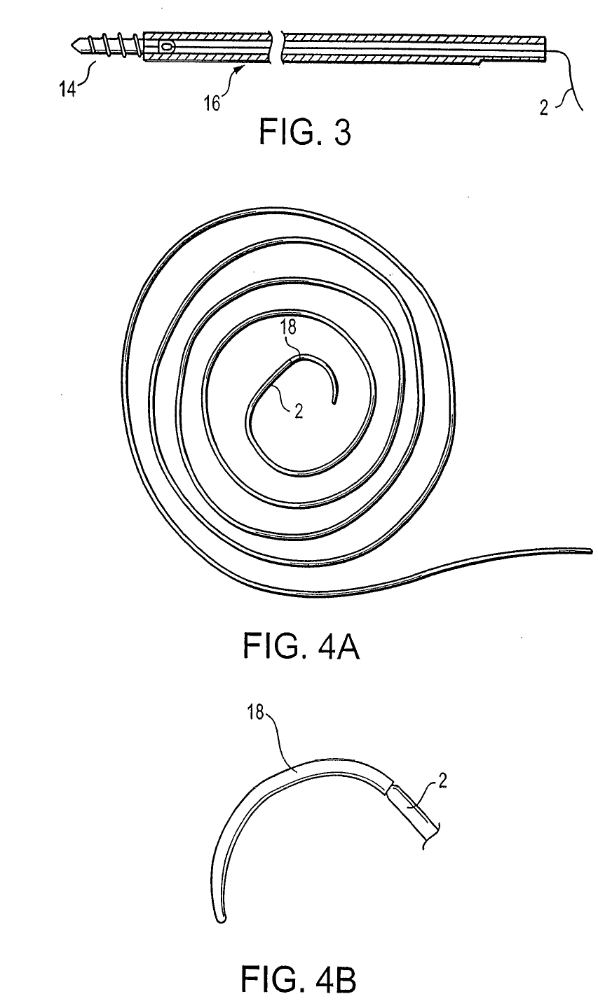

In one method of using the suture of the present invention, the suture 2 is attached to a suture anchor 14 as shown in FIG. 3 (prepackaged sterile with an inserter 16), or is attached at one or both ends to a half round, tapered needle 18 as shown in FIGS. 4A and 4B.

As set forth in Example 1 as described above, one or more strands of a fiber in the blend can be provided in pre-dyed colors, e.g., black, to provide a trace. The trace threads enhance the ability to visually detect suture motion and the ability to differentiate between colored and uncolored suture strands.

Although the present invention has been described in relation to particular embodiments thereof, many other variations and modifications and other uses will become apparent to those skilled in the art. It is preferred, therefore, that the present invention be limited not by the specific disclosure herein, but only by the appended claims.

We claim:

1. A suture strand, comprising:
a cover including a plurality of fibers of ultrahigh molecular weight polyethylene and a plurality of fibers of polybutester; and
a core including a plurality of braided fibers of ultrahigh molecular weight polyethylene, the core surrounded by the cover.

2. The suture strand of claim 1, wherein the core comprises about three strands of ultrahigh molecular weight polyethylene twisted at about three to six twists per inch.

3. The suture strand of claim 1, wherein the cover comprises about eight strands of ultrahigh molecular weight polyethylene braided with about six strands of polybutester and about two strands of nylon.

4. The suture strand of claim 1, wherein the ultrahigh molecular weight polyethylene comprises at least 40% of the braided fibers.

5. The suture strand of claim 1, wherein the core comprises about 18% or greater of total amount of filament.

6. The suture strand of claim 1, further comprising a coating disposed on the cover, the coating being selected from the group consisting of wax, silicone, silicone rubbers, PTFE, PBA, and ethyl cellulose.

7. The suture strand of claim 1, wherein polybutester fibers in the cover are coated with a polytribolate polymer.

8. A suture assembly comprising:
a suture having a longitudinal length and a multifilament cover formed of a plurality of braided fibers of ultrahigh molecular weight polyethylene and fibers of polybutester, and a core formed of twisted fibers of ultrahigh molecular weight polyethylene; and
a suture anchor attached to the suture.

9. A suture assembly comprising:
a suture having a longitudinal length and a multifilament cover formed of a plurality of braided fibers of ultrahigh molecular weight polyethylene and fibers of polybutester, and a core formed of twisted fibers of ultrahigh molecular weight polyethylene; and
a half round, tapered needle attached to one or both ends of the suture.

10. A method of using a suture strand, comprising the steps of:
forming a multifilament suture strand having a longitudinal length and a multifilament cover formed of a plurality of braided fibers of ultrahigh molecular weight polyethylene and fibers of polybutester, and a core formed of twisted fibers of ultrahigh molecular weight polyethylene; and
attaching the suture strand to a suture anchor.
